(19) United States
(12) Patent Application Publication
(10) Pub. No.: US 2024/0390172 A1
(43) Pub. Date: Nov. 28, 2024

(54) MODIFICATION METHOD AND TOOL FOR QUICKLY RETROFITTING ELECTRONIC HARDWARE ON ORTHOSIS

(71) Applicant: ZHEJIANG UNIVERSITY, Hangzhou (CN)

(72) Inventors: Guanyun WANG, Hangzhou (CN); Yue YANG, Hangzhou (CN); Lei REN, Hangzhou (CN); Chuang CHEN, Hangzhou (CN); Ye TAO, Hangzhou (CN); Lingyun Sun, Hangzhou (CN)

(21) Appl. No.: 18/735,148

(22) Filed: Jun. 5, 2024

Related U.S. Application Data
(63) Continuation of application No. PCT/CN2023/089890, filed on Apr. 21, 2023.

(30) Foreign Application Priority Data
Apr. 10, 2023 (CN) 202310374095.2

(51) Int. Cl. A61F 5/01; H01R 13/62

(52) U.S. Cl. CPC A61F 5/0118; A61F 5/0125; H01R 13/6205

(57) ABSTRACT

A modification method and a tool for quickly retrofitting electronic hardware on an orthosis. The method mainly comprises drilling holes in an existing orthosis and embedding a magnetic connector or a conductive fabric electrode, ironing a circuit on the stereoscopic surface of the orthosis, and fixing a magnetic electronic functional module on the orthosis through magnetic attraction with the magnetic connector to realize circuit communication. The method is realized by a tool kit, which comprises a drilling tool, a circuit ironing tool, a magnetic connector, a magnetic electronic functional module, a wire tape and a conductive fabric electrode. This method can flexibly and quickly transform the orthosis according to the needs, with a strong applicability, and does not require the user to have circuitry expertise, which can shorten the customization and development cycle of the smart orthoses.

### CROSS-REFERENCE TO RELATED APPLICATIONS

The present application is a continuation of International Application No. PCT/CN2023/089890, filed on Apr. 21, 2023, which claims priority to Chinese Application No. 202310374095.2, filed on Apr. 10, 2023, the contents of both of which are incorporated herein by reference in their entireties.

### TECHNICAL FIELD

The present disclosure belongs to the technical field of object modification, and relates to a modification method and a tool for quickly retrofitting electronic hardware on an orthosis. The present disclosure can reconstruct rehabilitation orthoses with different materials, curvatures and thicknesses, and can quickly embed electronic equipment on the existing rehabilitation orthoses to realize the functions of illness monitoring and the like.

### BACKGROUND

Orthosis is an external support device aimed at alleviating the dysfunction of limbs, spine and skeletal muscle system, which is widely used in the field of medical rehabilitation. With the development and popularization of rehabilitation-related sensory monitoring and electronic therapy technologies, it has become an innovative product with great prospects in the field of smart medical care to increase the perception and interaction ability of orthoses. Smart orthoses can feedback the treatment effect in real time, offering accurate health data for rehabilitation doctors to meet the needs of patients for rehabilitation training, treatment assistance, and health monitoring.

However, the current high customization threshold of smart orthoses makes it difficult to popularize them in hospitals and daily rehabilitation settings for patients.

The specific reasons are as follows: firstly, the customization and development cycle of smart orthoses is relatively long, requiring consideration of the cooperation between the shape and structure of orthoses and the functional layout of electronic hardware in the early stage of orthosis design. Moreover, the development and manufacture of intelligent functions need a certain threshold of electronic technology, making it challenging for rehabilitation doctors without electronic professional skills to quickly produce and test smart orthoses for patients in hospitals or orthosis studios, potentially missing the golden treatment period for patients. Secondly, the use cycle of the orthoses is limited due to the changes in the rehabilitation stage and patients' physical condition. However, the high customization cost of the smart orthoses with electronic hardware makes it difficult for patients to accept the high-cost smart orthoses solutions. Thirdly, in some functional scenes that need electrodes to monitor skin signals closely, such as electrical stimulation and electromyography, hydrogel electrodes are commonly used in current rehabilitation monitoring and treatment equipment, which require accurate placement and frequent replacement, making it difficult for patients wearing the orthoses to perform daily operations. Fourthly, balancing the stability and flexibility of hardware is challenging: at present, the smart orthoses still in the research and development stage have exposed protruding wires, which cannot remain stability during patients' daily wear and activities. However, the fully encapsulated functional module cannot be flexibly adjusted according to the needs of patients and the different structural forms of orthoses.

### SUMMARY

In view of the shortcomings of the prior art, the object of the present disclosure is to provide a modification method and a tool for quickly retrofitting electronic hardware on an orthosis. By providing modular electronic components and installation tools, the modification method can enable an orthosis technician who has no professional background in electronic circuits to quickly and flexibly transform the existing orthosis through simple operations such as drilling, ironing and magnetic attraction, and retrofit customized sensing or therapeutic functions to the existing orthosis. The electronic components installed by the method can be quickly disassembled and reused through magnetic connection, thereby reducing the cost for a single patient. For functional scenes requiring electrodes, the conductive fabric electrode designed by this method can work stably on the orthosis for a long time without the need for patients to frequently put on and take off the orthosis and replace the electrode, and can be quickly installed by tapping or pressing, so as to keep stable contact with the skin. The circuit ironing tool provided by this method can quickly realize stable and attached circuit connection on the three-dimensional surface of the orthosis, thus ensuring wearing comfort and functional stability. The whole modification method is suitable for orthoses with different materials, thicknesses and three-dimensional shapes that are commonly available in the market, and can be extended to the intelligent modification of other products.

The technical solution adopted by the present disclosure is as follows:

The present disclosure relates to a modification method for quickly retrofitting electronic hardware on an orthosis, which is implemented by a tool kit; the tool kit includes a drilling tool, a circuit ironing tool, a magnetic connector, a magnetic electronic functional module, a wire tape and a conductive fabric electrode. The method includes the following steps:
S1, determining a type and an installation position of an electronic function to be retrofitted according to treatment or monitoring requirements of a patient;
S2, puncturing, by the drilling tool, on the orthosis at the determined installation portion, and embedding, by a patch, the magnetic connector at a portion where the magnetic electronic functional module is mounted.
S3, ironing, by the circuit ironing tool, the wire tape on a surface of the orthosis according to a preset wire layout.
S4, mounting the magnetic electronic functional module on the magnetic connector at a required portion of the orthosis, and when mounting the conductive fabric electrode, quickly mounting the conductive fabric electrode at a required hole portion to communicate a preset circuit, and completing a rapid modification of the orthosis.

Further, the drilling tool is configured to drill on the orthosis to embed the magnetic connector or the conductive fabric electrode, the circuit ironing tool is configured to iron and attach the wire tape to the surface of the orthosis; the magnetic electronic functional module is provided at the magnetic connector, and the magnetic electronic functional module and the magnetic connector are firmly connected through magnetic attraction; electric communication between the magnetic electronic functional module and the wire tape is achieved through cooperation between the magnetic electronic functional module and the magnetic connector; and the wire tape is configured to communicate different magnetic electronic functional modules or conductive fabric electrodes to form required functional circuits.

Further, the circuit ironing tool includes an ironing head, a heating core and a heat-insulating handle shell. The heating core is arranged in the heat-insulating handle shell, and a front end of the heating core extends out and is connected to a metal plate, and the metal plate is wrapped with a soft flame-retardant heat-conducting material to form the ironing head, and the heating core is an electric heating wire.

Further, the heat-insulating handle shell of the circuit ironing tool is further provided with a rotary feeding structure and a cutting head, the wire tape is coiled in the rotary feeding structure, and the cutting head is located at an outlet of the rotary feeding structure.

Further, the wire tape is a three-layer composite structure consisting of a conductive layer, an intermediate layer and a carrier layer. The conductive layer is a conductive wire flat cable arranged at a lowest portion, the intermediate layer is a thermal bonding layer, and the carrier layer is a release paper; and the conductive layer, the intermediate layer and the carrier layer are bonded together by heating, and windows are provided at intervals along a length direction of the wire tape, and the intermediate layer and the carrier layer are not arranged at the windows, allowing the conductive layer expose.

Further, the magnetic electronic functional module includes a magnet, a functional circuit board and a pin terminal thereof, and the magnetic connector includes a magnet and a wiring terminal; both the pin terminal and the wiring terminal are provided with at least two types, namely, the magnetic electronic functional module and the magnetic connector have at least two types; the pin terminal in a first type magnetic electronic functional module is a spring thimble pin, and the wiring terminal in a first type magnetic connector is a flat or convex contact. The spring thimble pin corresponds to the flat or convex contact; the pin terminal in a second type magnetic electronic functional module is a hole-shaped contact, the wiring terminal in a second type magnetic connector is a spring thimble pin, and a back of the second type magnetic connector is correspondingly provided with a flat or convex contact. The hole-shaped contact corresponds to the spring thimble pin, and the spring thimble pin is communicated with the flat or convex contact.

Further, when the magnetic electronic functional module needs to be installed on an outer surface of the orthosis, the first type magnetic connector and the first type magnetic electronic functional module are selected; after the first type magnetic connector is installed at a corresponding hole position of the orthosis and the wire tape is ironed, the first type magnetic electronic functional module is tightly attracted to the first type magnetic connector by magnetic attraction, so that the spring thimble pin abuts against the flat or convex contact and the wire tape is sandwiched therebetween, and a contact position between the wire tape and the spring thimble pin is a window for exposing a wire thereof, so that the first type magnetic electronic functional module is connected with the wire; when the magnetic electronic functional module needs to be installed on an inner surface of the orthosis, the second type magnetic electronic functional module and two second type magnetic connectors are selected; after one second type magnetic connector is installed in a corresponding hole position of the orthosis and the wire tape is ironed, the second type magnetic electronic functional module is tightly attracted to the second type magnetic connector through magnetic attraction so as to insert the spring thimble pin into the hole-shaped contact, the other second type magnetic connector is tightly attracted to the previous second type magnetic connector through magnetic attraction, so that the spring thimble pin abuts against the flat or convex contact and the wire tape is sandwiched therebetween; and the wire in the wire tape contacts with the flat or convex contact, so that the second type electronic functional module is communicate with the wire.

Further, the conductive fabric electrode includes a conductive fabric, an annular foam cotton pad, a snap fastener rivet and an annular gasket; the snap fastener rivet and the annular gasket are both conductive materials; a rivet female nail is fixed at a center of the annular foam cotton gasket, the conductive fabric wraps the annular foam cotton gasket to form a part A of the conductive fabric electrode, and the annular gasket is sleeved at a rivet male pin to form a part B of the conductive fabric electrode; the part A and the part B are fixedly connected by fastening the rivet female nail with the rivet male pin, the annular gasket is pressed against a window portion of the wire tape during fastening, and the wire is exposed at window portion.

Further, after the modified orthosis is used by the patient, the magnetic electronic functional module, the magnetic connector and the conductive fabric electrode can be quickly removed and reused on other orthoses.

The present disclosure has the advantages that:

The method of the present disclosure lowers the threshold for personalized customization of the intelligent orthosis, eliminating the need for lengthy development cycles, allowing orthotists without electronic backgrounds in welding and programming to directly customize personalized electronic functions on the ready-made orthosis quickly, to flexibly determine the location layout of hardware circuits according to the patient's condition, and quickly complete the modification, testing and delivery on the spot. Modular functional components can be recycled quickly and reused flexibly, reducing the high cost. The electrode remain stable without frequent replacement, ensuring stable monitoring or therapeutic functions while the orthosis is worn continuously. The whole modification method is applicable to commonly available orthoses of different materials, thicknesses and three-dimensional shapes, facilitating the widespread adoption of personalized manufacturing for smart orthoses.

### BRIEF DESCRIPTION OF DRAWINGS

FIG. 1 is an overall schematic diagram of the method flow in the present disclosure.

FIGS. 2(a) and 2(b) show a schematic structural diagram of the circuit ironing tool.

FIG. 3(a) is ae flow chart of using the circuit ironing tool, and FIG. 3(b) is a structural schematic diagram of a wire tape.

FIG. 4(a) is an example schematic diagram of a first type magnetic electron attracting functional module, and FIG. 4(b) is a second type magnetic electron attracting functional module.

FIG. 5(a) is a schematic structural view of the magnetic electronic functional module installed on the outer surface of the orthosis, and FIG. 5(b) is the inner surface of the orthosis.

FIG. 6(a) is a schematic structural diagram of the conductive fabric electrode in the pre-buckled state, and FIG. 6(b) is a schematic structural diagram of the conductive fabric electrode in the buckled state.

FIGS. 7(a), 8(b), 8(c) and 8(d) show a flow chart of the installation and manufacture of the conductive fabric electrode.

FIGS. 8(a), 8(b), 8(c), 8(d) and 8(e) show a schematic diagram of retrofitting a pressure monitoring function to a hand orthosis, by taking the hand thermoplastic orthosis as an example.

FIGS. 9(a), 9(b), 9(c), 9(d), 9(e), 9(f), 9(g1) and 9(g2) show a schematic diagram of retrofitting motion angle monitoring and EMG signal measurement e functions to the fixed knee orthosis by taking the dynamic knee orthosis as an example.

FIGS. 10(a), 10(b), 10(c) and 10(d) show a schematic diagram of retrofitting a muscle electrical stimulation function to a patella fixing orthosis by taking the patella fixing orthosis as an example.

FIGS. 11(a1), 11(a2), 11(b1), 11(b2), 11(c1) and 11(c2) show a schematic diagram of ironing electronic circuits on different surfaces and structures by using ironing tools. The flexibility of the ironing angle and the shape adaptability of the ironing head can allow the wire tape to conform to complex geometric surfaces.

FIGS. 12(a), 12(b), 12(c) and 12(d) show a test diagram of embedding electronic equipment in different materials, curvatures and thicknesses by using the method of the present disclosure.

FIGS. 13(a), and 13(b) show that the mechanical arm simulation test and outdoor wear and tear test are used to evaluate the stability of the intelligent orthosis made by the method of the present disclosure.

Reference signs: 1. Ironing head; 2. heating core; 3. heat-insulating handle shell; 4. rotary feeding structure; 5. Cutting head; 6. Conductive layer; 7. Window; 8. Stamp hole; 9. Magnet; 10. Spring thimble pin; 11. Hole-shaped contact; 12. Mark; 13. First type magnetic electronic functional modules; 14. Second type magnetic electronic functional modules; 15. First type magnetic connector; 16. Second type magnetic connectors; 17. Wire tape; 18. Patch; 19. Hole position on the orthosis; 20. Flat or convex contact; 21. Rivet male pin; 22. Rivet female nail; 23. Annular gasket; 24. Orthosis; 25. Annular foam cotton gasket; 26. Conductive fabric; 27. Skin; 28. Thermal adhesive layer.

### DESCRIPTION OF EMBODIMENTS

Next, the technical solution of the present disclosure will be further described in detail with reference to the attached drawings and specific examples.

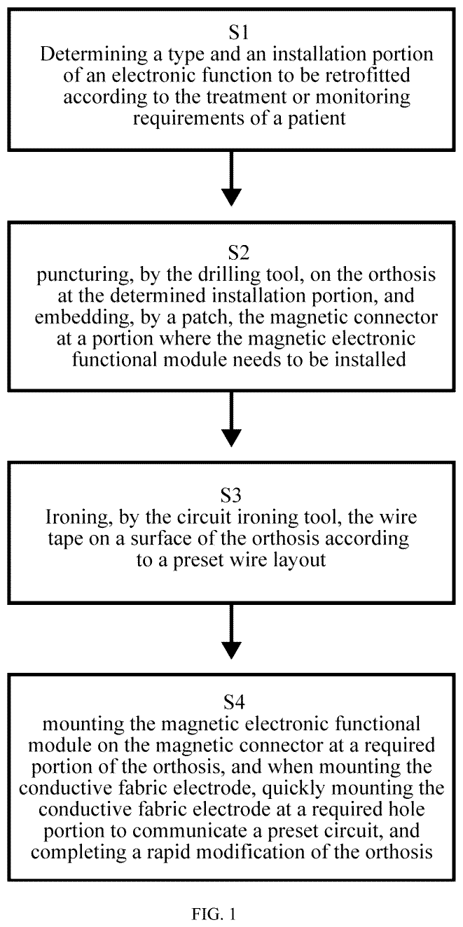

The present disclosure relates to a modification method and a tool for quickly retrofitting electronic hardware on an orthosis. According to a specific example of the present disclosure, the method is realized by a tool kit, which mainly includes a drilling tool, a circuit ironing tool, a magnetic connector, a magnetic electronic functional module, a wire tape and a conductive fabric electrode; this method is suitable for orthoses with different materials, different thicknesses and complex geometric surfaces in the market. The main flow of the method includes the following (as shown in FIG. 1):
S1, the type and the installation position of the electronic equipment to be retrofitted are determined according to the treatment or monitoring requirements of a patient.
S2, a hole is drilled on the existing orthosis by using a drilling tool at the position where an electronic functional module needs to be installed, and a magnetic connector is embedded through a patch; a hole is drilled at a position where an electrode needs to be installed by using the drilling tool for installing a conductive fabric electrode.
S3, a wire tape is ironed on the surface of the existing orthosis according to a certain layout by using a circuit ironing tool.
S4, the magnetic electronic functional module is installed on the magnetic connector at the required position of the orthosis (a user can quickly connect the modules according to the marks such as arrows on the surface of the magnetic electronic functional module) to complete the modification of the orthosis.
S5, after use, the electronic equipment (such as the magnetic electronic functional module, the magnetic connector and the conductive fabric electrode) can be quickly recovered and reused on other orthoses.

In the tool kit used in the method, the drilling tool is used for drilling holes on the orthosis to embed magnetic connectors or conductive fabric electrodes, the circuit ironing tool is used for ironing and attaching the wire tape to the surface of the orthosis, and the magnetic electronic functional module is installed on the magnetic connector, and the magnetic electronic functional module and the magnetic connector are firmly connected through magnetic attraction, so that the electric communication between the magnetic electronic functional module and the wire tape is realized through the cooperation of the magnetic electronic functional module and the magnetic connector; the wire tape is used to connect different magnetic electronic functional modules or conductive fabric electrodes to form required functional circuits.

In the flow S1, firstly, the specific functions to be retrofitted to the orthosis are determined, and electronic components with corresponding capabilities are selected; for example, if the user wants a pressure sensing function to be installed, a pressure monitoring module is selected. In an example of the present disclosure, the electronic functional modules used for basic functional modification include a power module, a controller module and one or more sensor modules. In addition, output electronic modules (lighting and vibration) for interaction can be additionally selected. After selecting the electronic modules, the user temporarily places the module on the orthosis according to the series structure of the circuit modules (such as power supply→controller→sensor) to plan the sensor layout and determine the drilling positions. It is recommended to give priority to planning the positions of important sensors, for example arranging a pressure sensor near a key pressure point of the orthosis, and then planning the positions of other components and circuit layout.

In the flow S2, a customized thermal drilling tool is adopted in the present disclosure, and the thermal drilling tool for installing the magnetic connector and the conductive fabric electrode has a drill bit with a sufficient length (greater than 18 mm), which can penetrate orthosis materials with different thicknesses; the thermal drilling tool has two replaceable bit modules with different shapes to meet different drilling requirements. The cost is reduced by sharing a heating source, and the bit module may be replaced by a stud bolt structure. Among them, the drill bit with a round port matches the profile of the metal rivet buckle of the conductive fabric electrode (about 3 mm), and the rounded rectangular drill bit matches the profile of the magnetic connector.

Considering the modular length of the ironing circuit, this method provides a flexible ruler with holes to help plan the layout of components and determine the drilling position of the magnet connector. The hole of the flexible ruler is slightly larger than the magnetic connector, allowing the drilling tool head to pass through. The flexible ruler has the same elasticity and turning radius (63 mm) as the ironing circuit.

In an embodiment, corresponding to the drill bit, the magnetic connector in the thermal drilling tool has a positioning thimble higher than the edge plane of the outer contour in its central area, which is convenient for accurate positioning at the preset drilling position.

For magnetic electronic functional modules with different pin numbers, it is necessary to drill different numbers of holes to realize different numbers of magnetic connector shapes. Magnetic electronic functional modules with more pins (two rows of pins less than 8) can be installed on the double holes drilled by the thermal drilling tool, and electronic equipment with less pins (one row of pins less than 4) can be installed on the single holes drilled by the thermal drilling tool or on any row of the double holes.

A rounded rectangular patch with an area larger than that of the magnetic connector is stuck on the surface of the magnetic connector and the orthosis, so that the magnetic connector and the orthosis are fixed, and the patch can be set as a hollow structure according to needs, so that the magnetic electronic functional module and the magnetic connector can be closely attached, and the patch can strengthen the stability of the magnetic connection of electronic equipment and improve the aesthetics of the modified orthosis.

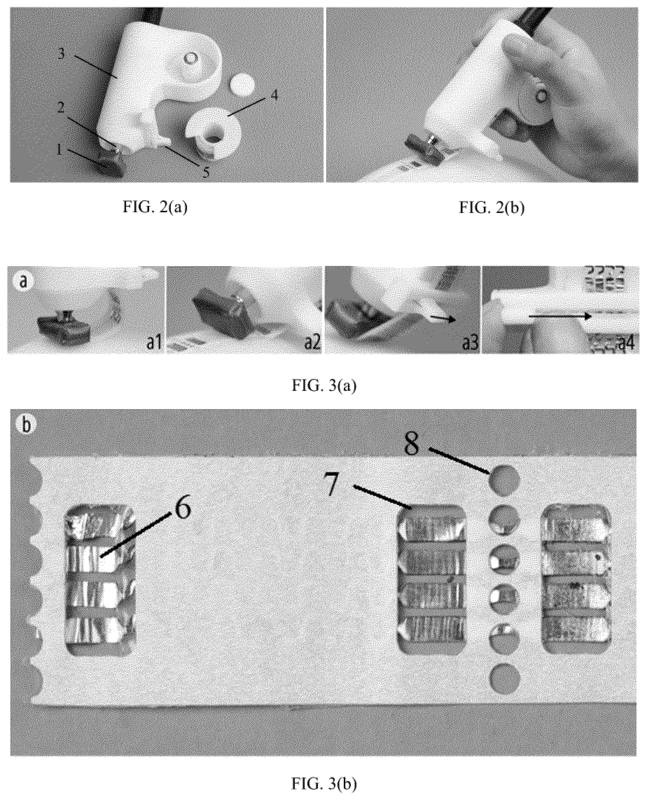

In the flow S3, as shown in FIGS. 2(a) and 2(b), the circuit ironing tool includes an ironing head, a heating core and a heat-insulating handle shell. The heating core is arranged in the heat-insulating handle shell, the front end of the heating core extends out and is connected with a metal plate, and a soft flame-retardant heat-conducting material wraps the outside the metal plate to form the ironing head, and the heating core is an electric heating wire. A rotary feeding structure and a cutting head can further be arranged, the wire tape are coiled in the rotary feeding structure, and the cutting head is located at the outlet of the rotary feeding structure. In this example, the circuit ironing tool consists of a heat-insulating handle, a copper tape consumable roll, a heating wire of an electric soldering iron and an ironing head. The heat-insulating handle is made of 3D printed ABS material. In order to prevent the heat-insulating handle from being heated to cause deformation of the shell, the inner heating wire is wrapped with a heat-insulating material (5 mm-thick aluminum silicate refractory fiber felt). In order to ensure the thermal insulation between the electric soldering iron and the user, the distance between the ABS shell of the ironing tool and the thermal insulation material is about 5 mm. The copper tape is wound on the replaceable bobbin, and the circuit consumables in the ironing tool can be easily and quickly replaced. The ironing tool is modified on the basis of the electric soldering iron equipment, and the original welding head is replaced by a customized metal plate, which is connected and fixed by an M4 screw. The welding head has a soft hot rubber pad (2 mm thick) and Teflon tape to prevent adhesion to the low-temperature thermoplastic material.

Because the ironing head in the circuit ironing tool is wrapped with soft flame-retardant and heat-conductive material, it can closely fit the three-dimensional curved surface of the orthosis by pressing down, and the angle between the ironing head and the surface of the orthosis can be flexibly adjusted during use, so that the circuit can be ironed on the curved surface of the orthosis. As shown in FIG. 3(a), the user's specific operation steps are as follows: after preheating the ironing tool with a copper wire circuit tape, first the circuit starting point is aligned with the magnetic connector on the orthosis and ironed for fixation, the tool is moved on the surface of the orthosis, the angle of the circuit ironing tool is flexibly adjusted to ensure that the circuit routing direction passes through the magnetic connector, the soft ironing head is slightly pressed to fit the different geometric shapes of the orthosis, and the circuit traces are ironed on the curved surface; in this example, the wire tape is a copper tape, and the TPU layer in the copper tape becomes sticky at the temperature of 120 to 150° C., which can stick the circuit to the orthosis. Before ironing, the cutting head is slid on the ironing orthosis to make the inner cutting blade cut off the copper tape, and finally the end of the copper tape is ironed onto the magnetic connector.

For a shorter circuit path, users may choose to cut the copper tape directly and finish ironing quickly on the curved surface of the orthosis; for a longer circuit path, the user may choose to use the rotary feeding structure of the circuit ironing tool, and the copper tape can pass through the gap of the heat-insulating handle to the position of the ironing head; the circuit ironing tool equipped with a copper tape can be ironed quickly on the three-dimensional curved surface, and the copper tape can be conveniently supplemented during ironing, so as to realize rapid wiring in the scene where a long circuit connection is needed.

The wire tape in the present disclosure is a three-layer composite structure consisting of a conductive layer, an intermediate layer and a carrier layer. The conductive layer is a conductive wire flat cable arranged at the bottom, the intermediate layer is a thermal bonding layer, and the carrier layer is a release paper, and the three layers are bonded into a whole by heating; windows are arranged at intervals along the length direction of the wire tape, and the intermediate layer and the carrier layer are not arranged at the windows, so that the conductive layer is exposed. In the above example, the copper tape ironed on the surface of the orthosis consists of three layers: the bottom layer is the conductive layer, and the copper foil with conductive adhesive is cut into serpentine lines, allowing the circuit to stretch to adapt to the curved surface of the modified orthosis and the slight deformation that may exist during the use of the orthosis; the middle layer is an adhesive and protective layer, and a thermoplastic polyurethane (TPU) film with hot melt adhesive can be stably adhered to various surface materials of the orthoses (such as ABS, PCL, sponge and fabric) after heating, and in addition, TPU can increase the resistance to water and dust intrusion, and can withstand the wear and tear of long-term daily use (such as the friction of nylon belt); the top layer is to prevent the adhesive and protective layer from sticking to the ironing tool during ironing, and a release paper is added to the top of the copper tape and can be torn off after the circuit is ironed.

The production of the copper tape is as follows: the conductive layer of the copper tape can be made by cutting copper foil with a laser marking machine, the adhesive layer and the carrier layer can be made by cutting TPU and release paper with a laser cutting machine, and the three layers can be bonded by an ironing tool.

As shown in FIG. 3(b)—, in order to realize the communication between the upper and lower surfaces while protecting the circuit, a row of laser-cut hollow holes are formed on the TPU layer at intervals (for example, 3.5 cm) to form windows, corresponding to the positions where the pins or metal contacts of the electronic functional modules and the magnetic attraction sensors; there is a row of laser-cut stamp holes corresponding to each interval, which is convenient for quick cutting with the cutting head (cutting slider) of the circuit ironing tool. The circuit of the modified orthosis can be customized in a unit of 3.5 cm.

In the flow S4, the magnetic electronic functional module includes a magnet, a functional circuit board and a pin terminal thereof, and the magnetic connector includes a magnet and a wiring terminal; both the pin terminal and the wiring terminal are provided with at least two types, namely, the magnetic electronic functional module and the magnetic connector have at least two types; the pin terminal in a first type magnetic electronic functional module is a spring thimble pin, and the wiring terminal in a first type magnetic connector is a flat or convex contact. The spring thimble pin corresponds to the flat or convex contact; the pin terminal in a second type magnetic electronic functional module is a hole-shaped contact, the wiring terminal in a second type magnetic connector is a spring thimble pin, and a back of the second type magnetic connector is correspondingly provided with a flat or convex contact. The hole-shaped contact corresponds to the spring thimble pin, and the spring thimble pin is communicated with the flat or convex contact. The connection mechanism between the electronic functional module and the orthosis ensures the stability and durability of the modified orthosis in the use process. The circuit connection structure realized by using the method of the present disclosure is as follows:

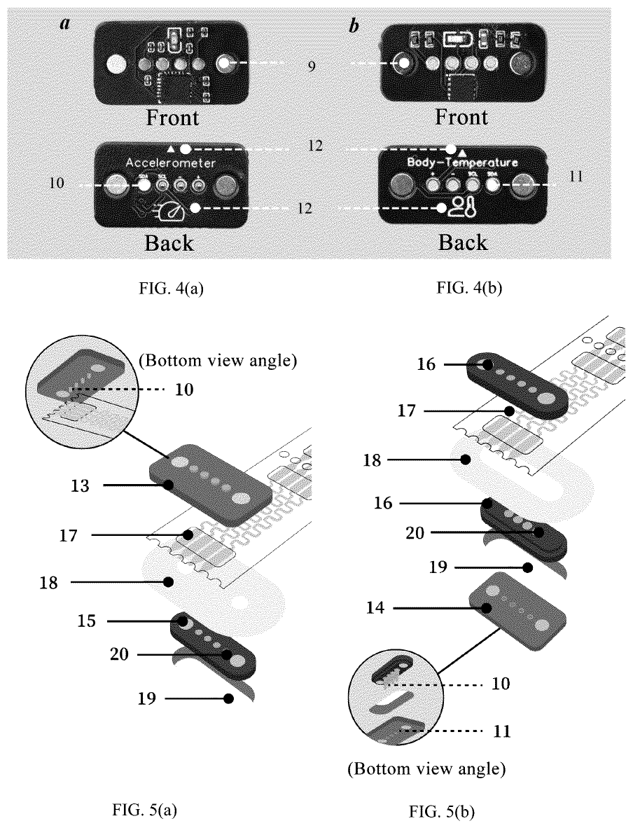

For electronic equipment that needs to be installed on the outer surface of the orthosis, a magnetic connector with a patch is embedded in the orthosis, which provides magnetic force for quickly installing electronic components (such as power supply, controller and acceleration sensor module) on the outer surface of the orthosis. As shown in FIG. 4(a) and FIG. 5a, when a magnetic electronic functional module needs to be installed on the outer surface of the orthosis, a first type magnetic connector and a first type magnetic electronic functional module are selected. After the first type magnetic connector is installed in the corresponding hole position of the orthosis and the wire tape is ironed, the first type magnetic electronic functional module is tightly attracted to the first type magnetic connector by magnetic attraction, so that the spring thimble pin is pressed against the flat or convex contact and the wire tape is sandwiched therebetween, and the contact position between the wire tape and the spring thimble pin is a window for exposing the wire thereof, thereby allowing the magnetic electronic functional module to be communicated with the wire; in the present disclosure, the customized first type magnetic electronic functional module can be provided with a magnet and a short spring thimble, which can be simply installed and disassembled for recycling or maintenance, and at the same time, possible falling off during use can be avoided. The circuit ironed on the surface of the orthosis is sandwiched between the magnetic connector and the spring thimble of the magnetic electronic functional module. The elastic spring thimble can ensure that the electronic circuit ironed on the surface (where the wire tape has a window) can be touched to ensure reliable and stable electrical connection.

When the magnetic electronic functional module needs to be installed on the inner surface of the orthosis, one second type magnetic electronic functional module and two second type magnetic connectors are selected, and after one second type magnetic connector is installed in the corresponding hole position of the orthosis and the wire tape is ironed, the second type magnetic electronic functional module is tightly attracted to the second type magnetic connector through magnetic attraction to insert the spring thimble pin into the hole-shaped contact; the other second type magnetic connector is tightly attracted to the previous second type magnetic connector through magnetic attraction, so that the pin of the spring thimble is pressed against the flat or convex contact and the wire tape is sandwiched therebetween, and the wire in the wire tape contacts with the flat or convex contact, so that the second type electronic functional module is communicate with the wire. For example, sensors (such as body temperature sensor, heart rate blood oxygen sensor, humidity sensor and membrane pressure sensor) that must be installed inside the orthosis and close to the body should be connected to the circuit on the outer surface of the orthosis. As shown in FIG. 5(b), a magnetic connector with a patch and a long spring thimble (for example, the compression range is from 3.8 mm to 5.3 mm) is embedded in the orthosis. The long spring thimble can penetrate the orthoses with different thicknesses, hardness and slight curvatures, and cab be connected to the hole pin (hole-shaped contact) of the sensor inside the orthosis to form an electrical connection. A controller module or a magnetic connector with a short spring thimble is installed on the circuit trace on the outer surface of the orthosis to ensure the connection stability. In addition, a layer of sponge will be laid around the inner sensor to ensure the wearing comfort of the patient.

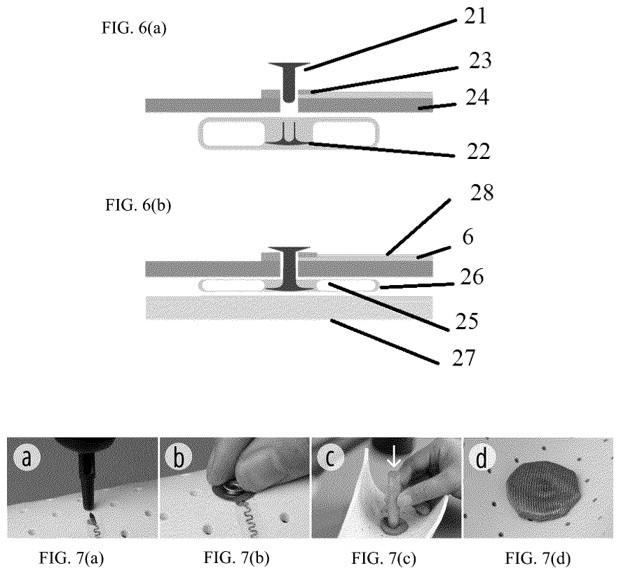

For EMG sensors and electrical stimulation modules, conductive fabric electrodes can be installed for direct contact with the human body to exchange physiological electrical signals. As shown in FIGS. 6(a) and 6(b), the conductive fabric electrode has the following structural characteristics: the conductive fabric electrode includes conductive fabric, an annular foam cotton gasket, a snap fastener rivet and an annular gasket; both the rivet and the annular gasket are conductive materials; a rivet female nail is fixed in the center of the annular foam cotton gasket, and the conductive fabric wraps the annular foam cotton gasket to form a part A of the conductive fabric electrode; the annular gasket is sleeved on a rivet male pin to form a part B of the conductive fabric electrode; the parts A and B are fixedly connected by fastening of the rivet female nail and the rivet male pin, and the fastening requires the annular gasket to be pressed against a window position of the wire tape, where the wire thereof is exposed. As shown in FIGS. 7(a), 7(b), 7(c) and 7(d), the electrode installation process is as follows: the user first drills a small hole at the end of the line at a required position, then puts an annular copper gasket and rivet male pin (part B) outside the orthosis, and puts a conductive fabric electrode with rivet female nail (part A) inside, and then fixes the inner and outer rivets by knocking, so as to realize the stable contact and connection of the conductive fabric, the snap fastener rivet, the copper gasket and the circuit line, thus realizing the installation of the fabric electrode. A LCR digital bridge test showed that the contact impedance of the conductive fabric electrode designed by the present disclosure is equivalent to that of the commercial hydrogel electrode, and provides user comfort and convenience superior to that of the commercial electrode.

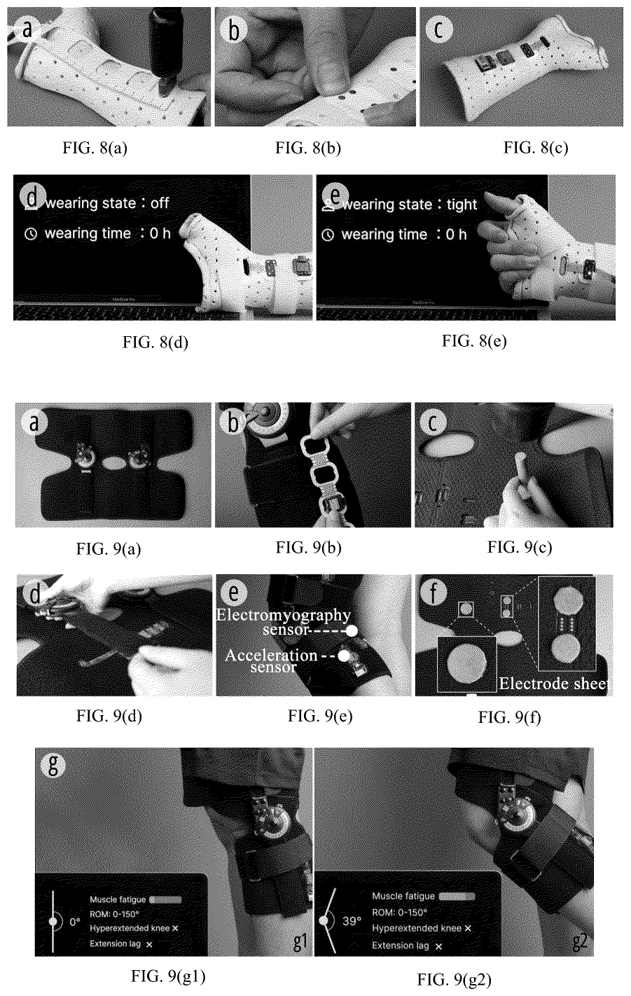

As shown in FIGS. 8(a), 8(b), 8(c) 8(d) and 8(e), as a specific embodiment of the application of the present disclosure, a customized wrist orthosis for treating wrist fracture is embedded with a pressure sensor for monitoring the wearing state and duration of the patient. A thin film pressure sensor is attached to the inner surface of the bony protrusion which is prone to excessive pressure. When the orthosis is too tight or too loose, a LED indicator module on the orthosis will remind the wearer by flashing. In the process of rehabilitation, the electronic component module can be quickly removed and installed on different modified orthoses.

As shown in FIGS. 9(a), 9(b), 9(c), 9(d), 9(e), 9(f), 9 (g1) and 9 (g2), as a concrete embodiment of the application of the present disclosure, an acceleration sensor and an electromyography sensor are embedded in a dynamic knee orthosis for treating ligament fracture and protecting meniscus, so as to detect and remind the patient of the range of motion and the degree of muscle fatigue. It can prevent the secondary injury caused by excessive exercise and quickly evaluate the range of motion of the knee joint of the patient, so that the patient is able to independently and safely complete daily rehabilitation training without the on-site supervision of doctors.

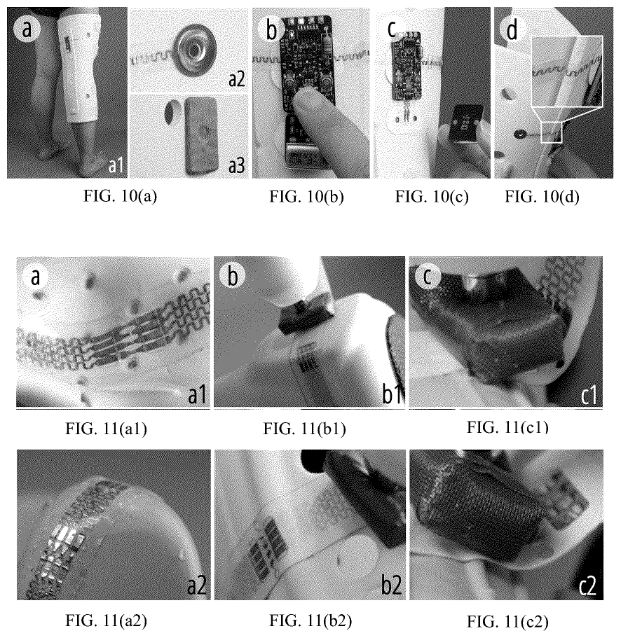

As shown in FIGS. 10(a), 10(b), 10(c) and 10(d), as a concrete embodiment of the application of the present disclosure, an electric stimulation module is embedded into a patella fixation orthosis for treating patella fracture to prevent muscle atrophy, and the patient can be treated with safe electric stimulation at home by using the intelligent orthosis. Conductive fabric electrodes are embedded in the patella fracture orthosis, and the externally installed power module is convenient for disassembly and replacement, and can be charged in time. The circuit formed by copper tape ironing has stability and will not be broken by the deformation of the foam splint of the orthosis.

The method of the present disclosure is applicable for the surface of the orthosis with a complex geometric surface. As shown in FIGS. 11(a1), 11(a2), 11(b1), 11(b2), 11(c1) and 11(c2), due to the flexibility of the ironing angle and the shape adaptability of the ironing head, the circuit can be ironed on the orthoses with different complex geometries, for example the curved surfaces of the orthoses corresponding to the depression of the palm and the lump of the ankle, the local thickening position of the orthosis and the reinforcing ribs.

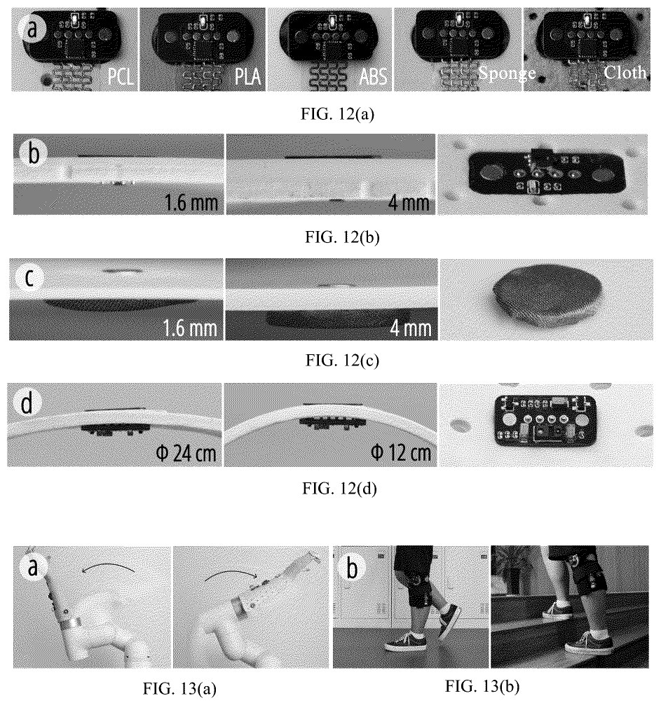

The method of the present disclosure is applicable for transforming common orthosis materials, as shown in FIG. 12(a), and the adaptable materials include but are not limited to low-temperature thermoplastic plates, PLA, ABS, sponges, cloth and other common materials for rehabilitation orthoses.

The method of the present disclosure is applicable for the surfaces of orthoses with different thicknesses. As shown in FIGS. 12(a) and (b), due to the tolerance of the interval between the magnetic connector and the spring thimble, the method of the present disclosure can realize the electrical and mechanical connection between the inside and outside of the orthoses for orthosis materials with different thicknesses, orthoses with different sponge linings, and orthoses with undulating shapes. The thickness of a typical orthosis ranges from 1.6 mm to 4 mm, and an extra 3 mm-thick foam pad will be stuck on the inside of the orthosis to smooth the edge of the electronic equipment inside. The total length of the long spring thimble of the magnetic connector is 5.3 mm, and the maximum compression is 3.8 mm. When the material of the orthosis is thinner than 3.8 mm, the foam pad will cover the gap between the orthosis and the electronic equipment, ensuring the comfortable fit while maintaining the electrical and mechanical connection. When the material thickness of the orthosis is greater than 5.3 mm, a longer spring thimble can be used to increase the applicable thickness range. If a stronger magnetic force is needed, a magnet can be added between the magnetic connector and the electronic equipment. Because of the adaptability of rivets and foam cotton wrapping inside the conductive fabric electrode, the conductive fabric electrode may further adapt to different thicknesses (1.6 mm-4.0 mm).

According to the method, a mechanical arm simulation motion state wearing test and an outdoor wearing wear test prove that the modified intelligent orthosis has enough stability and is applicable for different daily use scenes. As shown in FIGS. 13(a), and 13(b), a wrist orthosis after intelligent modification is swung back and forth by 120° every 3 seconds by a mechanical arm to simulate the movement of a patient during long-term use. The mechanical arm test shows that all electronic components work well, and the 50-mAh battery is exhausted after about 3 hours and more than 7200 swings. The intelligent wrist orthosis can continue to work normally after the battery module is quickly replaced. In addition, the intelligent modified dynamic knee orthosis was further tested outdoors for five days to evaluate the mechanical durability and electrical continuity under real use conditions. The test shows that the intelligent orthosis can ensure the stability and continuity of the electronic equipment under daily use by patients.

This method has the characteristics of simple and efficient operation, compatibility with different curved surface shapes and thicknesses, and flexible adjustment and reuse of components. The method of modifying the orthosis may further be migrated and extended to other fields that need to be retrofitted with electronic hardware, such as sports equipment (such as helmets) and household products.

What is claimed is:

1. A modification method for quickly retrofitting electronic hardware on an orthosis, wherein the modification method is implemented by a tool kit, and the tool kit comprises a drilling tool, a circuit ironing tool, a magnetic connector, a magnetic electronic functional module, a wire tape and a conductive fabric electrode; and the method comprises:
step S1, determining a type and an installation portion of an electronic function to be retrofitted according to treatment or monitoring requirements of a patient;
step S2, puncturing, by the drilling tool, on the orthosis at the determined installation portion, and embedding, by a patch, the magnetic connector at a portion where the magnetic electronic functional module is mounted;
step S3, ironing, by the circuit ironing tool, the wire tape on a surface of the orthosis according to a preset wire layout; and
step S4, mounting the magnetic electronic functional module on the magnetic connector at a required portion of the orthosis, and when mounting the conductive fabric electrode, quickly mounting the conductive fabric electrode at a required hole portion to communicate a preset circuit, and completing a rapid modification of the orthosis.

2. The modification method according to claim 1, wherein the drilling tool is configured to drill on the orthosis to embed the magnetic connector or the conductive fabric electrode, the circuit ironing tool is configured to iron and attach the wire tape to the surface of the orthosis; the magnetic electronic functional module is provided at the magnetic connector, and the magnetic electronic functional module and the magnetic connector are firmly connected through magnetic attraction; electric communication between the magnetic electronic functional module and the wire tape is achieved through cooperation between the magnetic electronic functional module and the magnetic connector; and the wire tape is configured to communicate different magnetic electronic functional modules or conductive fabric electrodes to form required functional circuits.

3. The modification method according to claim 1, wherein the circuit ironing tool comprises an ironing head, a heating core and a heat-insulating handle shell, and wherein the heating core is arranged in the heat-insulating handle shell, and a front end of the heating core extends out and is connected to a metal plate, and the metal plate is wrapped with a soft flame-retardant heat-conducting material to form the ironing head, and the heating core is an electric heating wire.

4. The modification method according to claim 3, wherein the heat-insulating handle shell of the circuit ironing tool is further provided with a rotary feeding structure and a cutting head, the wire tape is coiled in the rotary feeding structure, and the cutting head is arranged at an outlet of the rotary feeding structure.

5. The modification method according to claim 1, wherein the wire tape is a three-layer composite structure comprising a conductive layer, an intermediate layer and a carrier layer, and wherein the conductive layer is a conductive wire flat cable and arranged at a lowest portion, the intermediate layer is a thermal bonding layer, and the carrier layer is a release paper; and the conductive layer, the intermediate layer and the carrier layer are bonded together by heating, and windows are provided at intervals along a length direction of the wire tape, and the intermediate layer and the carrier layer are not arranged at the windows, allowing the conductive layer expose.

6. The modification method according to claim 1, wherein the magnetic electronic functional module comprises a magnet, a functional circuit board and a pin terminal, and the magnetic connector comprises a magnet and a wiring terminal; both the pin terminal and the wiring terminal are provided with at least two types, namely, the magnetic electronic functional module and the magnetic connector have at least two types; the pin terminal of a first type magnetic electronic functional module is a spring thimble pin, and the wiring terminal of a first type magnetic connector is a flat or convex contact, wherein the spring thimble pin corresponds to the flat or convex contact; the pin terminal of a second type magnetic electronic functional module is a hole-shaped contact, the wiring terminal of a second type magnetic connector is a spring thimble pin, and a back of the second type magnetic connector is correspondingly provided with a flat or convex contact, and wherein the hole-shaped contact corresponds to the spring thimble pin, and the spring thimble pin is communicated with the flat or convex contact.

7. The modification method according to claim 6, wherein when the magnetic electronic functional module is mounted at an outer surface of the orthosis, the first type magnetic connector and the first type magnetic electronic functional module are selected; after the first type magnetic connector is mounted at a corresponding hole portion of the orthosis and the wire tape is ironed, the first type magnetic electronic functional module is tightly attracted to the first type magnetic connector by magnetic attraction, so that the spring thimble pin abuts against the flat or convex contact and the wire tape is sandwiched between the spring thimble pin and the flat or convex contact, and a contact portion between the wire tape and the spring thimble pin is a window for exposing a wire, so that the first type magnetic electronic functional module is connected to the wire; when the magnetic electronic functional module is mounted at an inner surface of the orthosis, the second type magnetic electronic functional module and two second type magnetic connectors are selected; after one second type magnetic connector is mounted at the corresponding hole portion of the orthosis and the wire tape is ironed, the second type magnetic electronic functional module is tightly attracted to the second type magnetic connector through magnetic attraction to insert the spring thimble pin into the hole-shaped contact, the other second type magnetic connector is tightly attracted to the previous second type magnetic connector through magnetic attraction, so that the spring thimble pin abuts against the flat or convex contact, and the wire tape is sandwiched between the spring thimble pin and the flat or convex contact; and the wire in the wire tape contacts with the flat or convex contact, so that the second type electronic functional module is communicate with the wire.

8. The modification method according to claim 1, wherein the conductive fabric electrode comprises a conductive fabric, an annular foam cotton pad, a snap fastener rivet and an annular gasket; the snap fastener rivet and the annular gasket are both conductive materials; a rivet female nail is fixed at a center of the annular foam cotton gasket, the conductive fabric wraps the annular foam cotton gasket to form a part A of the conductive fabric electrode, and the annular gasket is sleeved at a rivet male pin to form a part B of the conductive fabric electrode; the part A and the part B are fixedly connected by fastening the rivet female nail with the rivet male pin, the annular gasket is pressed against a window portion of the wire tape during fastening, and the wire is exposed at window portion.

9. The modification method according to claim 1, wherein after the modified orthosis is used by the patient, the magnetic electronic functional module, the magnetic connector and the conductive fabric electrode is quickly removed and reused on other orthoses.

10. A tool for quickly retrofitting electronic hardware on an orthosis, wherein the tool is the tool kit in the modification method according to claim 1.
